(19) United States
(12) Patent Application Publication
(10) Pub. No.: US 2025/0346236 A1
(43) Pub. Date: Nov. 13, 2025

(54) SYSTEM AND METHOD FOR DETERMINING IMPAIRED DRIVING

(71) Applicant: Quanata, LLC, San Francisco, CA (US)

(72) Inventor: Gil Tamari, San Francisco, CA (US)

(73) Assignee: Quanata, LLC, San Francisco, CA (US)

(21) Appl. No.: 18/661,406

(22) Filed: May 10, 2024

(51) Int. Cl. B60W 40/08; A61B 5/00; A61B 5/18; G06Q 30/0207; G06Q 40/08; H04W 4/02

(52) U.S. Cl. CPC B60W 40/08; A61B 5/18; A61B 5/4845; G06Q 30/0207; G06Q 40/08; H04W 4/02; B60W 2040/0845; B60W 2540/22

(57) ABSTRACT

A method including receiving sensor data from one or more substance-detecting sensors located near or on a user is disclosed. The one or more substance-detecting sensors can be configured to detect one or more chemical substances. The method further can include determining, based on the sensor data, one or more consumed substances of the one or more chemical substances consumed by the user. The method also can include determining, in real-time, whether the user is driving under an influence of the one or more consumed substances. The method additionally can include updating a user profile of the user to include user driving behavior data comprising one or more of: (a) the sensor data, (b) the one or more consumed substances, or (c) a determination that the user is driving under influence of the one or more consumed substances. Other embodiments are disclosed.

### FIELD OF THE DISCLOSURE

The present disclosure generally relates to technologies for determining impaired driving.

### BACKGROUND

Drivers influenced by certain substances while driving vehicles can be difficult to identify without field sobriety tests. Even when signs of impairment are shown, existing tests cannot reliably identify all of the substances used. For example, among the common causes of impaired driving, marijuana (or more specifically, tetrahydrocannabinol (THC)) can be tested by breath within 2 hours or by blood or saliva within 4 hours of use, while the effect of marijuana on driving can last up to 6 hours or more. Therefore, systems and methods for easily and reliably determining potential impaired driving caused by marijuana or similar substances are desired.

### BRIEF DESCRIPTION OF THE DRAWINGS

The figures described below depict various aspects of the systems and methods disclosed therein. It should be understood that each figure depicts an embodiment of a particular aspect of the disclosed systems and methods, and that each of the figures is intended to accord with a possible embodiment thereof. Further, wherever possible, the following description refers to the reference numerals included in the following figures, in which features depicted in multiple figures are designated with consistent reference numerals.

There are shown in the drawings arrangements which are presently discussed, it being understood, however, that the present embodiments are not limited to the precise arrangements and are instrumentalities shown, wherein:

FIG. 1 illustrates a front elevation view of a computer system that is suitable for implementing an exemplary embodiment of the system disclosed in FIG. 3;

FIG. 2 illustrates a representative block diagram of an example of the elements included in the circuit boards inside a chassis of the computer system of FIG. 1;

FIG. 3 illustrates a system for determining impaired driving, according to one exemplary embodiment; and

FIG. 4 illustrates a flow chart for a method for determining a user's driving is impaired by substances consumed prior to or while driving, according to one exemplary embodiment.

The figures depict preferred embodiments for purposes of illustration only. One skilled in the art will readily recognize from the following discussion that alternative embodiments of the systems and methods illustrated herein can be employed without departing from the principles of the invention described herein.

### DETAILED DESCRIPTION OF THE DRAWINGS

The present embodiments can generally relate to, inter alia, using a non-invasive method to remotely determine whether a user is driving under the influence of certain substances (e.g., marijuana or alcohol). In particular, the detection or determination of a user's consumption of these substances before or while driving can provide valuable information for determining whether the user drives safely for a trip, and can form an important basis, or a portion thereof, for improving courses of action by entities, such as price changes, product or service changes or modifications, company policy changes impacting employees, reward policy changes, a police officer's decisions as to whether or not to initiate an impaired-driving investigation, etc.

More specifically, various embodiments can include a method for determining, in real-time, whether a driver is impaired by drugs (e.g., marijuana, alcohol, etc.) that is consumed before or at the time of driving. The method can include: (a) receiving sensor data from one or more substance-detecting sensors located near or on a user, wherein the one or more substance-detecting sensors are configured to detect one or more chemical substances; (b) determining, based on the sensor data, one or more consumed substances of the one or more chemical substances consumed by the user; (c) determining, in real-time, whether the user is driving under an influence of the one or more consumed substances; and (d) updating a user profile of the user to include user driving behavior data comprising one or more of: (i) the sensor data, (ii) the one or more consumed substances, or (iii) a determination that the user is driving under influence of the one or more consumed substances. The method can include additional, less, or alternate functionality, including that discussed elsewhere herein.

In one aspect, a system for determining impaired driving can be provided. The computer system can include one or more local or remote processors, servers, sensors, memory units, transceivers, mobile devices, wearables, smart watches, smart rings, smart glasses or contacts, augmented reality glasses, virtual reality headsets, mixed or extended reality headsets, voice bots, chat bots, ChatGPT bots, InstructGPT bots, Codex bots, Google Bard bots, and/or other electronic or electrical components, which can be in wired or wireless communication with one another. For instance, in one aspect, the computer system can include one or more local or remote processors and/or associated transceivers; and one or more local or remote non-transitory computer-readable media storing computing instructions that, when run on the one or more processors, direct the one or more processors to perform one or more actions or operations.

The computing instructions can direct the systems and/or processor(s) to: (a) receive sensor data from one or more substance-detecting sensors located near or on a user, the one or more substance-detecting sensors being configured to detect one or more chemical substances; (b) determine, based on the sensor data, one or more consumed substances of the one or more chemical substances consumed by the user; (c) determine, in real-time, whether the user is driving under an influence of the one or more consumed substances; and (d) updating a user profile of the user to include user driving behavior data comprising one or more of: (i) the sensor data, (ii) the one or more consumed substances, or (iii) a determination that the user is driving under influence of the one or more consumed substances. The system can be configured to include additional, less, or alternate functionality, including that discussed elsewhere herein.

In another aspect, a computer readable storage medium storing computing instructions can be provided. The computing instructions, when run on one or more processors, can cause the one or more processors to: (a) receive sensor data from one or more substance-detecting sensors located near or on a user, the one or more substance-detecting sensors being configured to detect one or more chemical substances; (b) determine, based on the sensor data, one or more consumed substances of the one or more chemical substances consumed by the user; (c) determine, in real-time, whether the user is driving under an influence of the one or more consumed substances; and (d) updating a user profile of the user to include user driving behavior data comprising one or more of: (i) the sensor data, (ii) the one or more consumed substances, or (iii) a determination that the user is driving under influence of the one or more consumed substances. The computer readable storage medium can be configured to include additional, less, or alternate functionality, including that discussed elsewhere herein.

Advantages will become more apparent to those skilled in the art from the following description of the preferred embodiments which have been shown and described by way of illustration. As will be realized, the present embodiments can be capable of other and different embodiments, and their details are capable of modification in various respects. Accordingly, the drawings and description are to be regarded as illustrative in nature and not as restrictive.

Determining driving under the influence of certain substances, as mentioned above, can be based on sensor data received from sensors located near the user before or while the user is driving. Such a determination can be used by, for example, an insurance company to determine a discount value for an auto insurance policy of the user, law enforcement to stop unsafe driving, etc.

In many embodiments, the techniques described herein can provide a practical application and several technological improvements. The techniques described herein can provide a technical improvement to systems and/or methods for detecting impaired driving. In particular, the techniques described here can determine a user's use of marijuana and remotely monitor the user's behavior afterwards to identify impaired driving and its cause even after the testing window is closed. These techniques described herein can provide a significant improvement over conventional approaches that cannot identify impaired driving without field sobriety tests and also cannot reliably determine the substances that cause the impaired driving.

In certain aspects, a method can include receiving sensor data from one or more substance-detecting sensors located near or on a user. The one or more substance-detecting sensors can be configured to detect one or more chemical substances. The method further can include determining, based on the sensor data, one or more consumed substances of the one or more chemical substances consumed by the user. In addition, the method can include determining, in real-time, whether the user is driving under an influence of the one or more consumed substances. Moreover, the method can include updating a user profile of the user to include user driving behavior data comprising one or more of: (a) the sensor data, (b) the one or more consumed substances, or (c) a determination that the user is driving under influence of the one or more consumed substances.

### Exemplary Computer Systems

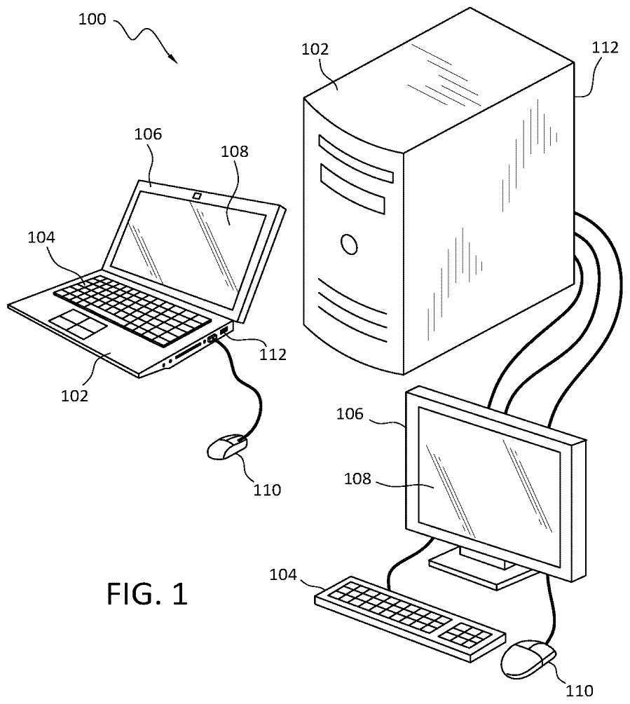

Turning to the drawings, FIG. 1 illustrates an exemplary embodiment of two different types (e.g., a laptop and a tower server) a computer system 100, all of which or a portion of which can be suitable for (i) implementing part or all of one or more embodiments of the techniques, methods, and systems and/or (ii) implementing and/or operating part or all of one or more embodiments of the non-transitory computer readable media described herein. As an example, a different or separate one of computer system 100 (and its internal components, or one or more elements of computer system 100) can be suitable for implementing part, or all of, the techniques described herein. Computer system 100 can comprise chassis 102 containing one or more circuit boards (not shown) and one or more of an input/output port 112 (e.g., one or more universal serial bus (USB) ports of one or more types (e.g., USB type-A, type-B, type-C, micro-A, micro-B, mini-A, mini-B, etc.), one or more High-Definition Multimedia Interface (HDMI) ports, etc.).

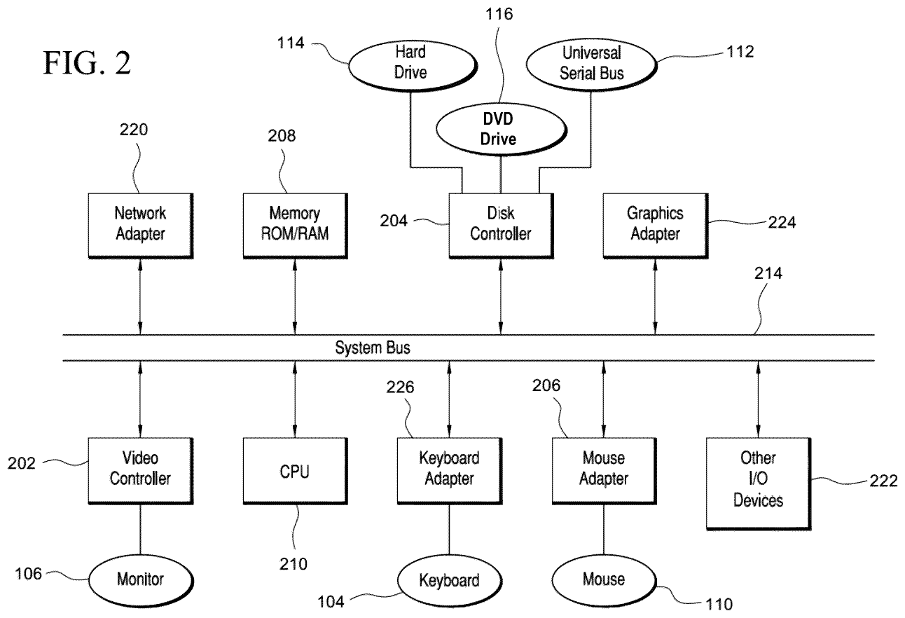

A representative block diagram of the elements included on the circuit boards inside chassis 102 is shown in FIG. 2. A central processing unit (CPU) 210 in FIG. 2 is coupled to a system bus 214. In various embodiments, the architecture of CPU 210 can be compliant with any of a variety of commercially distributed architecture families.

Continuing with FIG. 2, system bus 214 can also be coupled to memory storage unit 208 that includes both read only memory (ROM) and random access memory (RAM). Non-volatile portions of memory storage unit 208 or the ROM can be encoded with a boot code sequence suitable for restoring computer system 100 (FIG. 1) to a functional state after a system reset. In addition, memory storage unit 208 can include microcode such as a Basic Input-Output System (BIOS). In some examples, the one or more memory storage units of the various embodiments disclosed herein can include memory storage unit 208, a USB-equipped electronic device (e.g., an external memory storage unit (not shown) coupled to input/output port 112 (FIGS. 1-2)), hard drive 114 (FIG. 2), and/or one or more CD-ROM, DVD, Blu-Ray, or other suitable media, such as media configured to be used in CD-ROM and/or DVD drive 116 (FIG. 2) inside chassis 102 (FIG. 1) or in a detachable drive coupled to input/output port 112 (FIGS. 1-2).

Non-volatile or non-transitory memory storage unit(s) refer to the portions of the memory storage units(s) that are non-volatile memory and not a transitory signal. In the same or different examples, the one or more memory storage units of the various embodiments disclosed herein can include an operating system, which can be a software program that manages the hardware and software resources of a computer and/or a computer network. The operating system can perform basic tasks such as, for example, controlling and allocating memory, prioritizing the processing of instructions, controlling input and output devices, facilitating networking, and managing files. Exemplary operating systems can include one or more of the following: (i) Microsoft® Windows® operating system (OS) by Microsoft Corp. of Redmond, Washington, United States of America, (ii) Mac® OS X by Apple Inc. of Cupertino, California, United States of America, (iii) UNIX® OS, and (iv) Linux® OS.

Further exemplary operating systems can comprise one of the following: (i) the iOS® operating system by Apple Inc. of Cupertino, California, United States of America, (ii) the Blackberry® operating system by Research In Motion (RIM) of Waterloo, Ontario, Mayada, (iii) the WebOS operating system by LG Electronics of Seoul, South Korea, (iv) the Android™ operating system developed by Google, of Mountain View, California, United States of America, (v) the Windows Mobile™ operating system by Microsoft Corp. of Redmond, Washington, United States of America, or (vi) the Symbian™ operating system by Accenture PLC of Dublin, Ireland.

As used herein, “processor” and/or “processing module” means any type of computational circuit, such as but not limited to a microprocessor, a microcontroller, a controller, a complex instruction set computing (CISC) microprocessor, a reduced instruction set computing (RISC) microprocessor, a very long instruction word (VLIW) microprocessor, a graphics processor, a digital signal processor, or any other type of processor or processing circuit capable of performing the desired functions. In some examples, the one or more processors of the various embodiments disclosed herein can comprise CPU 210.

In the depicted embodiment of FIG. 2, various I/O devices such as a disk controller 204, a graphics adapter 224, a video controller 202, a keyboard adapter 226, a mouse adapter 206, a network adapter 220, and other I/O devices 222 can be coupled to system bus 214. Keyboard adapter 226 and mouse adapter 206 can be coupled to a keyboard 104 (FIGS. 1-2) and a mouse 110 (FIGS. 1-2), respectively, of computer system 100 (FIG. 1). While graphics adapter 224 and video controller 202 are indicated as distinct units in FIG. 2, video controller 202 can be integrated into graphics adapter 224, or vice versa in other embodiments. Video controller 202 is suitable for refreshing a monitor 106 (FIGS. 1-2) to display images on a screen 108 (FIG. 1) of computer system 100 (FIG. 1). Disk controller 204 can control hard drive 114 (FIG. 2), input/output port 112 (FIGS. 1-2), and CD-ROM and/or DVD drive 116 (FIG. 2). In other embodiments, distinct units can be used to control each of these devices separately.

In some embodiments, network adapter 220 can comprise and/or be implemented as a WNIC (wireless network interface controller) card (not shown) plugged or coupled to an expansion port (not shown) in computer system 100 (FIG. 1). In other embodiments, the WNIC card can be a wireless network card built into computer system 100 (FIG. 1). A wireless network adapter can be built into computer system 100 by having wireless communication capabilities integrated into the motherboard chipset (not shown), and/or implemented via one or more dedicated wireless communication chips (not shown), connected through a PCI (peripheral component interconnector) or a PCI express bus of computer system 100 (FIG. 1) or input/output port 112 (FIG. 1). In other embodiments, network adapter 220 can comprise and/or be implemented as a wired network interface controller card (not shown).

Although many other components of computer system 100 are not shown, such components and their interconnection are well known to those of ordinary skill in the art. Accordingly, further details concerning the construction and composition of computer system 100 and the circuit boards inside chassis 102 are not discussed herein.

When computer system 100 in FIG. 1 is running, program instructions stored on a USB drive in input/output port 112, on a CD-ROM or DVD in CD-ROM and/or DVD drive 116 (FIG. 2) or in the detachable drive coupled to input/output port 112 (FIGS. 1-2), on hard drive 114 (FIG. 2), or in memory storage unit 208 (FIG. 2) are executed by CPU 210 (FIG. 2). A portion of the program instructions, stored on these devices, can be suitable for carrying out all or at least part of the techniques described herein. In various embodiments, computer system 100 can be reprogrammed with one or more modules, system, applications, and/or databases, such as those described herein, to convert a general purpose computer to a special purpose computer.

For purposes of illustration, programs and other executable program components are shown herein as discrete systems, although it is understood that such programs and components can reside at various times in different storage components of computer system 100, and can be executed by CPU 210. Alternatively, or in addition to, the systems and procedures described herein can be implemented in hardware, or a combination of hardware, software, and/or firmware. For example, one or more application specific integrated circuits (ASICs) can be programmed to carry out one or more of the systems and procedures described herein. For example, one or more of the programs and/or executable program components described herein can be implemented in one or more ASICs.

Although computer system 100 is illustrated as a laptop computer or a tower server in FIG. 1, there can be examples where computer system 100 can take a different form factor while still having functional elements similar to those described for computer system 100. In some embodiments, computer system 100 can comprise a single computer, a single server, or a cluster or collection of computers or servers, or a cloud of computers or servers. Typically, a cluster or collection of servers can be used when the demand on computer system 100 exceeds the reasonable capability of a single server or computer. In certain embodiments, computer system 100 can comprise a portable computer, such as a laptop computer. In certain other embodiments, computer system 100 can comprise a mobile device, such as a smartphone, smart glasses, smart watch, wearable, virtual reality headset, augmented reality glasses, etc. In certain additional embodiments, computer system 100 can comprise an embedded system.

### Exemplary Computer Systems for Determining Impaired Driving

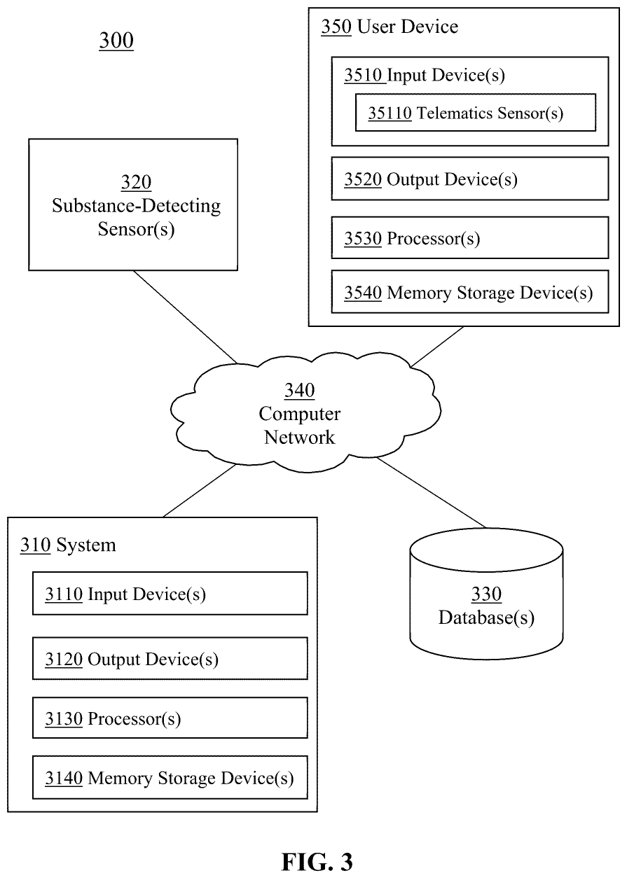

Turning ahead in the drawings, FIG. 3 illustrates a block diagram of a system 300 for determining, in real-time, whether the user is driving a vehicle while under the influence (DUI) of, or while being intoxicated (DWI) by, substances detected by sensors near the user, according to one embodiment. System 300 is exemplary, and embodiments of the system are not limited to the embodiments presented herein. The system can be employed in many different embodiments or examples not specifically depicted or described herein. In some embodiments, certain elements, modules, or systems of system 300 can perform various procedures, processes, operations, actions, and/or activities. In other embodiments, the procedures, processes, operations, actions, and/or activities can be performed by other suitable elements, modules, or systems of system 300.

Generally, therefore, system 300 can be implemented with hardware and/or software, as described herein. In some embodiments, part or all of the hardware and/or software can be conventional, while in these or other embodiments, part or all of the hardware and/or software can be customized (e.g., optimized) for implementing part or all of the functionality of system 300 described herein.

In some embodiments, system 300 can include one or more systems (e.g., a system 310), one or more substance-detecting sensors (e.g., substance-detecting sensor(s) 320), and/or one or more user devices (e.g., a user device 350) with sensors (e.g., telematics sensor(s) 35110). System 310 and user device 350 can each be a computer system, such as computer system 100 (FIG. 1), as described above, and can each be a single computer, a single server, or a cluster or collection of computers or servers, or a cloud of computers or servers. In another embodiment, a single computer system can host each of system 310 and user device 350.

In many embodiments, system 310 can be modules of computing instructions (e.g., software modules) stored on non-transitory computer readable media that operate on one or more processors. In other embodiments, system 310 can be implemented in hardware. In many embodiments, system 310 can comprise one or more systems, subsystems, modules, models, or servers. Additional details regarding system 310 and/or user device 350 are described herein.

In some embodiments, system 310 can be in data communication, through a computer network, a satellite network, a telephone network, or the Internet (e.g., computer network 340), with substance-detecting sensor(s) 320, and/or user device 350. In some embodiments, user device 350 can be used by users, such as users for system 310 and/or user device 350 (e.g., an insurance policyholder who signs up for DUI monitoring, etc.).

In certain embodiments, system 310 can host one or more websites and/or mobile application servers. For example, system 310 can host a website, or provide a server that interfaces with an application (e.g., a mobile application or a web browser), on user device 350, which can allow users to sign up for DUI monitoring, manage the user's profile, purchase a product, in addition to other suitable activities. In some embodiments, an internal network (e.g., computer network 340) that is not open to the public can be used for communications between system 310, substance-detecting sensor(s) 320, and/or user device 350 within system 300.

In many embodiments, user device 350 can include one or more input devices (e.g., input device(s) 3510), one or more output devices (e.g., output device(s) 3520), one or more processors (e.g., processor(s) 3530), and/or one or more memory storage devices (e.g., memory storage device(s) 3540). Examples of input device(s) 3510 can include one or more keyboards, one or more keypads, one or more pointing devices such as a computer mouse or computer mice, one or more touchscreen displays, a microphone, keyboard 104 (FIG. 1), mouse 110 (FIG. 1), etc. Examples of output device(s) 3520 can include one or more monitors, one or more touch screen displays, projectors, monitor 106 (FIG. 1), screen 108 (FIG. 1), etc. Examples of processor(s) 3530 can include CPU 210 (FIG. 2), etc. Examples of memory storage device(s) 3540 can include memory storage unit 208 (FIG. 2), external storage units coupled to input/output port 112 (FIGS. 1-2), hard drive 114 (FIG. 2), CD-ROM and/or DVD drive 116 (FIG. 2), a detachable drive coupled to input/output port 112 (FIGS. 1-2), etc. In a number of embodiments, input device 3510 further can include one or more sensors (e.g., telematics sensor(s) 35110, etc.).

Input device(s) 3510) and output device(s) 3520 can be coupled to user device 350 in a wired manner and/or a wireless manner, and the coupling can be direct and/or indirect, as well as locally and/or remotely. As an example of an indirect manner (which can or cannot also be a remote manner), a keyboard-video-mouse (KVM) switch can be used to couple input device(s) 3510 and output device(s) 3520 to processor(s) 3530 and/or memory storage device(s) 3540. In some embodiments, the KVM switch also can be part of user device 350. In a similar manner, processor(s) 3530 and/or memory storage device(s) 3540 can be local and/or remote to each other.

In certain embodiments, the user devices (e.g., user device 350) can be a mobile device, and/or other endpoint devices used by one or more users. A mobile device can refer to a portable electronic device (e.g., an electronic device easily conveyable by hand by a person of average size) with the capability to present audio and/or visual data (e.g., text, images, videos, music, etc.). For example, a mobile device can include at least one of a digital media player, a cellular telephone (e.g., a smartphone), a personal digital assistant, a handheld digital computer device (e.g., a tablet personal computer device), a laptop computer device (e.g., a notebook computer device, a netbook computer device), a wearable user computer device (e.g., smart glasses, smart watches, an augmented-reality (AR) headset, a virtual-reality (VR) headset, etc.), or another portable computer device with the capability to present audio and/or visual data (e.g., images, videos, music, etc.).

Thus, in many examples, a mobile device can include a volume and/or weight sufficiently small as to permit the mobile device to be easily conveyable by hand. For examples, in some embodiments, a mobile device can occupy a volume of less than or equal to approximately 1790 cubic centimeters, 2434 cubic centimeters, 2876 cubic centimeters, 4056 cubic centimeters, and/or 5752 cubic centimeters. Further, in these embodiments, a mobile device can weigh less than or equal to 15.6 Newtons, 17.8 Newtons, 22.3 Newtons, 31.2 Newtons, and/or 44.5 Newtons.

Exemplary mobile devices can include (i) an iPod®, iPhone®, iTouch®, iPad®, MacBook® or similar product by Apple Inc. of Cupertino, California, United States of America, (ii) a Blackberry® or similar product by Research in Motion (RIM) of Waterloo, Ontario, Mayada, (iii) a Lumia® or similar product by the Nokia Corporation of Keilaniemi, Espoo, Finland, and/or (iv) a Galaxy™ or similar product by the Samsung Group of Samsung Town, Seoul, South Korea. Further, in the same or different embodiments, a mobile device can include an electronic device configured to implement one or more of (i) the iPhone® operating system by Apple Inc. of Cupertino, California, United States of America, (ii) the Blackberry® operating system by Research In Motion (RIM) of Waterloo, Ontario, Mayada, (iii) the Android™ operating system developed by the Open Handset Alliance, or (iv) the Windows Mobile™ operating system by Microsoft Corp. of Redmond, Washington, United States of America.

In many embodiments, system 310 can include: (a) one or more input devices (e.g., input device(s) 3110 such as one or more keyboards, one or more keypads, one or more pointing devices such as a computer mouse or computer mice, one or more touchscreen displays, a microphone, etc.), (b) one or more display devices (e.g., output device(s) 3120 such as one or more monitors, one or more touch screen displays, projectors, etc.), (c) one or more processors (e.g., processor(s) 3130), and/or (d) one or more memory storage devices (e.g., memory storage device(s) 3540 such as one or more internal or external memory storage units, one or more hard drives, one or more CD-ROM or DVD drives, etc.). In these or other embodiments, one or more of the input device(s) (e.g., input device(s) 3110) can be similar or identical to keyboard 104 (FIG. 1) and/or a mouse 110 (FIG. 1). Further, one or more of the display device(s) (e.g., output device(s) 3120) can be similar or identical to monitor 106 (FIG. 1) and/or screen 108 (FIG. 1). Additionally, one or more of the processors (e.g., processor(s) 3130) can be similar or identical to CPU 210 (FIG. 2). In similar or different embodiments, one or more of the memory storage devices (e.g., memory storage device(s) 3140) can be similar or identical to memory storage unit 208 (FIG. 2), external storage units coupled to input/output 112 port (FIGS. 1-2), hard drive 114 (FIG. 2), CD-ROM and/or DVD drive 116 (FIG. 2), or a detachable drive coupled to input/output port 112 (FIGS. 1-2).

The input device(s) (e.g., input device(s) 3110) and the display device(s) (e.g., output device(s) 3120) can be coupled to system 310 in a wired manner and/or a wireless manner, and the coupling can be direct and/or indirect, as well as locally and/or remotely. As an example of an indirect manner (which can or cannot also be a remote manner), a keyboard-video-mouse (KVM) switch can be used to couple the input device(s) (e.g., input device(s) 3110) and the display device(s) (e.g., output device(s) 3120) to the processor(s) (e.g., processor(s) 3130) and/or the memory storage unit(s) (e.g., memory storage device(s) 3140). In some embodiments, the KVM switch also can be part of system 310. In a similar manner, the processors and/or the non-transitory computer-readable media can be local and/or remote to each other.

Meanwhile, in many embodiments, system 310 also can be configured to communicate with one or more databases (e.g., a database(s) 330). The one or more databases can include a member database that contains information about the demographic, geographic, and/or psychographic information of members of a population (e.g., insurance policyholders for an insurance company, etc.). The demographic, geographic, and/or psychographic information of the members can include the ages, genders, weights, residences, insurance policies, premiums, driving behavior data, payment history, and/or claim histories for the members, for example, among other information. The one or more databases additionally can include one or more of trained machine learning (ML) and/or artificial intelligence (AI) models (the ML/AI models) used in system 300 and/or system 310. The one or more databases also can include substance databases that contain information about substances involving impaired driving (e.g., alcohol, marijuana, opioids, other drugs, etc.). The information about driving related substances can include one or more sensors for detecting each substance at various levels of use or detection and optionally accounting for gender and weight of a person, time to reach a certain level of impairment or reach a non-impairment level, the legal level of impairment in various jurisdictions, the respective reaction time for each substance, the sensitivity of or the minimum level detectable by each sensor, etc. The one or more databases further can include training datasets for various ML/AI models, modules, or systems, etc. The training datasets can be obtained from a third party, generated manually, and/or curated from historical input/output data of one or more pre-trained ML/AI models, etc.

The one or more databases can be stored on one or more memory storage units (e.g., non-transitory computer readable media), which can be similar or identical to the one or more memory storage units (e.g., non-transitory computer readable media) described above with respect to computer system 100 (FIG. 1). Also, in some embodiments, for any particular database of the one or more databases, that particular database can be stored on a single memory storage unit or the contents of that particular database can be spread across multiple ones of the memory storage units storing the one or more databases, depending on the size of the particular database and/or the storage capacity of the memory storage units.

The one or more databases can each include a structured (e.g., indexed) collection of data and can be managed by any suitable database management systems configured to define, create, query, organize, update, and manage database(s). Exemplary database management systems can include MySQL (Structured Query Language) Database, PostgreSQL Database, Microsoft SQL Server Database, Oracle Database, SAP (Systems, Applications, & Products) Database, and IBM DB2 Database.

Meanwhile, system 300, system 310, and/or the one or more databases (e.g., database(s) 330) can be implemented using any suitable manner of wired and/or wireless communication. Accordingly, system 300 and/or system 310 can include any software and/or hardware components configured to implement the wired and/or wireless communication. Further, the wired and/or wireless communication can be implemented using any one or any combination of wired and/or wireless communication network topologies (e.g., ring, line, tree, bus, mesh, star, daisy chain, hybrid, etc.) and/or protocols (e.g., personal area network (PAN) protocol(s), local area network (LAN) protocol(s), wide area network (WAN) protocol(s), cellular network protocol(s), powerline network protocol(s), etc.). Exemplary PAN protocol(s) can include Bluetooth, Zigbee, Wireless Universal Serial Bus (USB), Z-Wave, etc.; exemplary LAN and/or WAN protocol(s) can include Institute of Electrical and Electronic Engineers (IEEE) 802.3 (also known as Ethernet), IEEE 802.11 (also known as WiFi), etc.; and exemplary wireless cellular network protocol(s) can include Global System for Mobile Communications (GSM), General Packet Radio Service (GPRS), Code Division Multiple Access (CDMA), Evolution-Data Optimized (EV-DO), Enhanced Data Rates for GSM Evolution (EDGE), Universal Mobile Telecommunications System (UMTS), Digital Enhanced Cordless Telecommunications (DECT), Digital AMPS (IS-136/Time Division Multiple Access (TDMA)), Integrated Digital Enhanced Network (iDEN), Evolved High-Speed Packet Access (HSPA+), Long-Term Evolution (LTE), WiMAX, etc.

The specific communication software and/or hardware implemented can depend on the network topologies and/or protocols implemented, and vice versa. In many embodiments, exemplary communication hardware can include wired communication hardware including, for example, one or more data buses, such as, for example, universal serial bus(es), one or more networking cables, such as, for example, coaxial cable(s), optical fiber cable(s), and/or twisted pair cable(s), any other suitable data cable, etc. Further exemplary communication hardware can include wireless communication hardware including, for example, one or more radio transceivers, one or more infrared transceivers, etc. Additional exemplary communication hardware can include one or more networking components (e.g., modulator-demodulator components, gateway components, etc.).

Still referring to FIG. 3, in many embodiments, system 310 can be configured to receive sensor data from one or more substance-detecting sensors (e.g., substance-detecting sensor(s) 320, telematics sensor(s) 35110, airborne-particle sensors, smoke detectors, etc.) located near a user in an enclosed space (e.g., a room, a vehicle, a public bathroom stall, etc.). In another embodiment, the sensor may be on a smart device such as a smart ring or smart watch or smart contact lens that can detect one or more substance via direct or indirect skin or body surface contact (e.g. fluid emitted (sweat saliva), light analysis (surface and penetrating), sound analysis (different substance resonates at different frequencies) or emitted odor, etc.). In this embodiment, a closed environment is not necessary and is optional. The one or more substance-detecting sensors can be configured to detect one or more chemical substances (e.g., alcohol, marijuana, inhalable cannabis-derived substances, etc.). The one or more substance-detecting sensors can be determined from one or more airborne-particle sensors based on the user location and a respective sensor location of each airborne-particle sensor. Moreover, system 310 can determine, in real-time, the user location of the user based at least in part on a mobile device of the user (e.g., user device 350). In a number of embodiments, system 310 further can be configured to determine, based on the sensor data, one or more consumed substances of the one or more chemical substances consumed by the user.

In many embodiments, system 310 also can determine, in real-time, whether the user is driving under an influence of the one or more consumed substances. To determine whether the user is driving under the influence of the one or more consumed substances, system 310 can determine: (a) whether the user is driving; and (b) whether the user is driving within a respective reaction time of at least one of the one or more consumed substances. Examples of the respective reaction time can include 4 hours, 6 hours, 8 hours, or 12 hours, after a consumed substance is consumed and detected. In another embodiment, the level of the substances detected may be used alone or in conjunction with reaction to determine impairment or reaction level. For example, a few jurisdictions can have zero tolerance laws that prohibits driving with any amount of drugs (e.g., THC and/or its metabolites) in the body (e.g., blood, saliva, breath, urine, etc.). Some jurisdictions can prohibit driving with a detectable amount of drugs in the body above a certain per se limit (e.g., 2 ng/ml, 5 ng/ml, etc. in blood; 0.5 μg/l, 1 μg/l, etc. in oral fluid; 35 pg/pad, 50 pg/pad, etc. in breath, etc.). Other jurisdictions can use both THC tests and field sobriety tests before imposing criminal sanctions on the tested drivers.

System 310 can determine whether the user is driving based at least in part on the user motion that can be determined based on one or more telematics sensors (e.g., telematics sensor(s) 35110) on: (a) a mobile device (e.g., user device 350) of the user, or (b) a user vehicle of the user. Examples of the telematics sensors for determining the user motion can include a Global-Positioning-System (GPS) unit, a vehicle speed sensor, a speedometer, etc. In certain embodiments, additional or alternative telematics sensors can be used to determine whether the user is driving. For example, a dash cam or an in-car security camera can be used with a vehicle speed sensor to detect whether the user is sitting behind the wheel of a moving vehicle.

In some embodiments, system 310 further can update a user profile of the user to include user driving behavior data. Example of the user driving behavior data can include: (a) the sensor data, (b) the one or more consumed substances, (c) a determination that the user is driving under influence of the one or more consumed substances, or (d) the combination thereof. In a number of embodiments, system 310 additionally can determine a discount for an insurance premium for the user based at least in part on the user driving behavior data.

### Exemplary Methods for Determining Impaired Driving

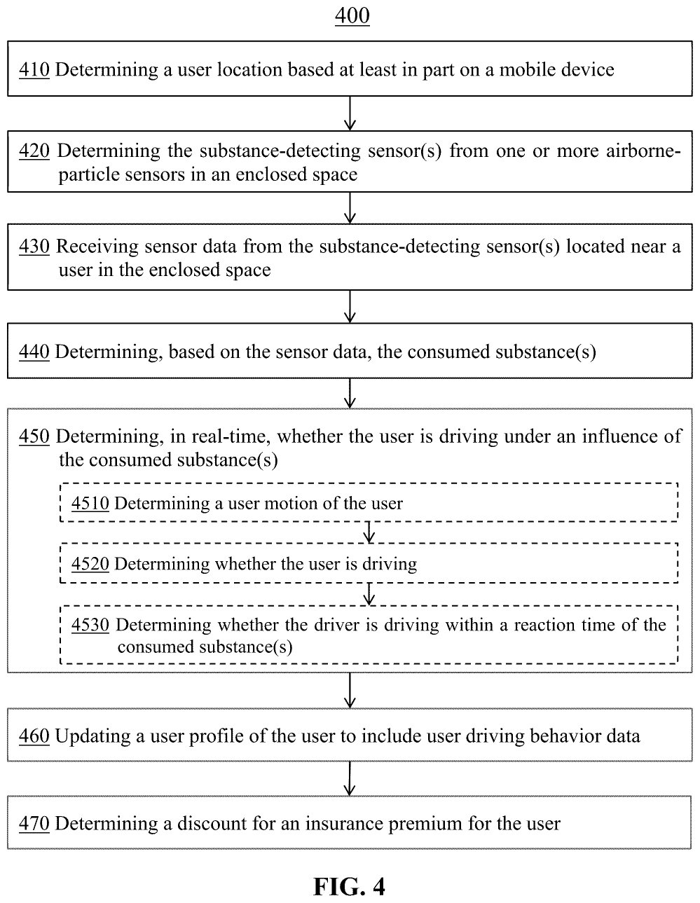

Turning ahead in the drawings, FIG. 4 illustrates actions of a method 400 for determining whether a user is driving under the influence of certain substances, according to certain embodiments. Method 400 can be implemented via execution of computing instructions configured to run on one or more processors and stored on one or more non-transitory computer-readable media. Method 400 is exemplary and is not limited to the embodiments presented herein. Method 400 can be employed in many different embodiments or examples not specifically depicted or described herein.

In some embodiments, the procedures, the processes, the operations, the actions, and/or the activities of method 400 can be performed in the order presented. In other embodiments, the procedures, the processes, the operations, the actions, and/or the activities of method 400 can be performed in any suitable order. In still other embodiments, one or more of the procedures, the processes, the operations, the actions, and/or the activities of method 400 can be combined or skipped.

In many embodiments, system 300 or system 310 (FIG. 3) (including one or more of its elements, modules, and/or systems, etc.) can be suitable to perform method 400 and/or one or more of the operations, actions, and/or activities of method 400. In these or other embodiments, one or more of the operations, actions, and/or activities of method 400 can be implemented as one or more computing instructions configured to run on one or more processors and configured to be stored on one or more non-transitory computer readable media. Such non-transitory computer readable media can be part of a computer system such as system 300 or system 310. The processor(s) can be similar or identical to the processor(s) described above with respect to computer system 100 (FIG. 1).

Referring to FIG. 4, in many embodiments, method 400 can include a block 410 of determining a user location based at least in part on a mobile device of the user (e.g., user device 350). For example, the user location can be determined based on the reading of the telematics sensor(s) 35110 (e.g., a GPS unit) of user device 350 that the user carries. In certain embodiments, the user location further can be determined based on one or more sensors (e.g., a GPS unit and/or a camera) on a vehicle.

In many embodiments, method 400 further can include a block 420 of determining the one or more substance-detecting sensors from one or more airborne-particle sensors (e.g., substance-detecting sensor(s) 320 (FIG. 3)) in an enclosed space. The one or more chemical substances to be detected can include one or more inhalable cannabis-derived substances. Each airborne-particle sensor can be configured to detect airborne particles of the one or more chemical substances in a user's breath. In some embodiments, each airborne-particle sensor for detecting whether the user consumed or is consuming the chemical substance(s) (e.g., alcohol or marijuana) can have a detection distance limit (e.g., 6 inches (in) or 15.24 centimeters (cm), 12 feet (ft) or 3.66 meters (m), etc.) and thus can be used only when the user is located within the limit. In similar or different embodiments, block 420 can select the one or more substance-detecting sensors based on the user location, as determined in block 410, and the respective location of each airborne-particle sensor. In a number of embodiments, the enclosed space can be substantially enclosed (e.g., a bedroom, a hotel room, an interior space of a vehicle, etc.) or partly enclosed (e.g., public bathroom stalls, etc.) as long as the airborne particles can be detected. Under certain circumstances, the enclosed space does not have to be enclosed per se but can include the near proximity (e.g., 5-8 in or 12.70-20.32 cm) of the user.

In a number of embodiments, method 400 also can include a block 430 of receiving, via computer network (e.g., computer network 340 (FIG. 3)), sensor data from the one or more substance-detecting sensors located near the user in the enclosed space, as determined at block 420. In still another embodiment, an enclosed space is not required, and detection may be through a wearable device such as a smart watch using direct or indirect detection methods (air, light, sound, etc.). In many embodiments, method 400 additionally can include a block 440 of determining, based on the sensor data, the one or more consumed substances. Generally, the presence of THC particles in the sensor data indicates the use of marijuana, and the presence of alcohol in the sensor data indicates alcoholic consumption.

In many embodiments, method 400 further can include a block 450 of determining, in real-time, whether the user is driving under an influence of, or while intoxicated by, the one or more consumed substances, determined at block 440. Block 450 further can include: (a) a block 4510 of determining a user motion of the user; (b) a block 4520 of determining whether the user is driving; and (c) a block 4530 of determining whether the driver is driving within a reaction time of at least one of the one or more consumed substances.

In a number of embodiments, block 4510 can determine the user motion of the user based on one or more telematics sensors (e.g., telematics sensor(s) 35110 (FIG. 3)) on: (a) a mobile device (e.g., user device 350 (FIG. 3)) of the user, and/or (b) a user vehicle of the user. Examples of the telematics sensors for detecting the user motion include a vehicle speedometer, an accelerometer on the mobile device, etc.

Block 4520 can determine whether the user is driving based at least in part on the user motion, determined at block 4510. For example, if the reading of telematics sensors show that the user is moving slowly (e.g., less than 30 miles per hour or 48.28 kilometers per hour), block 4510 can determine that the user is not driving. In another example, if the user is determined to be sitting in a backseat, instead of the driver seat, based on a picture taken, in real-time by an in-car security camera, then block 4510 further can determine that the user is not driving.

After it is determined at block 4520 that the user is driving, block 4530 can determine whether the driver is driving within a reaction time of at least one substance of the one or more consumed substances. For example, the reaction time, or time range, for marijuana can be 6-8 hours. If block 4530 determines that the driver is driving with the reaction time of marijuana that the user has used (as determined at block 440), block 450 can determine that the user is driving under the influence of the at least one substance (e.g., marijuana). In a few embodiments, the reaction time for a substance can be predetermined or determined, in real-time, by a trained machine learning (ML) or artificial intelligence (AI) model (an ML/AI model) based on the attributes of the substance and/or the information about the user (e.g., gender, ethnicity, lifestyle, health, etc.). Examples of the trained ML model can include decision trees, linear regressions, random forests, neural network models, etc., and/or the other ML models described herein.

In many embodiments, method 400 further can include a block 460 of updating a user profile of the user to include user driving behavior data. The user driving behavior data can include: (a) the sensor data, as received at block 430; (b) the one or more consumed substances, as determined at block 440; and/or (c) the determination that the user is driving under influence of the one or more consumed substances, as determined at block 450.

In several embodiments, method 400 further can include a block 470 of determining a discount for an insurance premium for the user based at least in part on the user driving behavior data, as updated at block 460. For example, after a user profile is updated in block 460 based on the user using consumer at least one of the consumed substances, the discount for the next insurance premium for the user may decrease.

In several embodiments, method 400 further can include additional or alternative processes, operations, acts, and/or activities. For example, method 400 further can include, before tracking the user location at block 410, providing a sign-up user interface with or without incentives to encourage users to participate in the DUI/DWI detection campaign. Method 400 also can include reporting, via emails or monthly statements, the DUI/DWI determination at block 450 or the update of the user profile at block 460, and so forth. Method 400 additionally can provide a survey to solicit user feedback, including the correctness of the DUI/DWI determination and/or explanations.

In a number of embodiments in which one or more ML/AI models are used (e.g., in some embodiments of block 4530), method 400 further can include pre-training and/or re-training the trained ML/AI models based upon historical input/output data, as determined in block 4530 respectively, feedback received from a user (e.g., the user's evaluation of the output, a new insurance claim associated with a trip monitored, etc.) and/or other data sources (e.g., DUI records of the user, etc.), and/or synthesized training data.

For each of the machine learning models to be retrained, the respective training datasets can be updated manually by a system user (e.g., an ML engineer, a data scientist, etc.) and/or automatically by a system (e.g., system 300 or 310 (FIG. 3)). The system user can select new training data from various data sources (e.g., websites, books, magazines, product catalogs, private third-party databases, etc.). The system can collect new training data based upon various criteria. In certain embodiments, historical input and/or output data of the model to be re-trained can be used for re-training the model. In several embodiments, the historical input and/or output data of the model can be selected based upon system performance and/or user feedback from the user associated with the historical output data.

### Exemplary Machine Learning Models

In many embodiments, the systems and/or methods can use one or more ML/AI models to perform one or more of the above-mentioned procedures, processes, activities, actions, operations, and/or methods. Further, the systems and/or methods can use one or more natural language processing (NLP) models for processing the one or more inputs and/or outputs (e.g., interpreting user feedback). Examples of the algorithms used for the various ML/AI models can include BERT, LLM, Lambda, Palm, XLNet, GPT-3, GPT-4, KNN, decision trees, linear regression, K-Means, neural networks, fuzzy logic, GANs, CTGAN, CNNs, VAEs, and so forth. In various embodiments, each of the ML/AI models used can be trained dynamically and/or regularly.

In many embodiments, the systems and/or methods can be configured to train or re-train the one or more ML/AI models. The training of each of the ML/AI models can be supervised, semi-supervised, and/or unsupervised, which in some embodiments can be followed by, or used in conjunction with, other techniques, such as re-enforcement machine learning techniques, or other techniques utilized by ChatGPT-based voice bots or virtual assistants. The training data of training datasets for pre-training or re-training each of the ML/AI models can be collected from various data sources, including historical input and/or output data by the ML/AI model. The collection and update of the training data in the training datasets can be performed once, periodically (e.g., every day, every week, etc.), or constantly. For example, in certain embodiments, the input and/or output data of an ML/AI model can be curated by a user (e.g., an ML engineer, a data scientist, etc.) or automatically collected every time the ML/AI model generates new output data to update the training datasets for re-training the ML/AI model. In many embodiments, the trained and/or re-trained ML/AI model as well as the training datasets can be stored in, updated, and accessed from a database (e.g., database(s) 330 (FIG. 3)).

In some embodiments, the systems, methods, and/or system users (e.g., a data scientist) further can determine whether to add the newly-created historical input and/or output data to the training dataset for retraining the ML/AI models based upon user feedback, predetermined criteria, and/or confidence scores for the historical output data. The user feedback can be associated with the output data of the ML/AI models or the output of the systems and/or methods using the ML/AI models.

In certain embodiments where machine learning techniques are not explicitly described in the processes, procedures, activities, operations, actions, and/or methods, such processes, procedures, activities, operations, actions, and/or methods can be read to include machine learning techniques suitable to perform the intended activities (e.g., determining, processing, analyzing, predicting, etc.). In several embodiments, the one or more ML/AI models can be configured to start or stop automatically upon occurrence of predefined events and/or conditions. In certain embodiments, the systems and/or methods can use a pre-trained ML/AI model, without any re-training.

### Additional Exemplary Embodiments for Detecting Impaired Driving

Various embodiments can include a method that can be implemented via execution of computing instructions configured to run on one or more processors and stored on one or more non-transitory computer-readable media. The method can include receiving sensor data from one or more substance-detecting sensors located near or on a user. The one or more substance-detecting sensors can be configured to detect one or more chemical substances. The one or more chemical substances can include one or more inhalable cannabis-derived substances.

In many embodiments, the method further can include determining, based on the sensor data, one or more consumed substances of the one or more chemical substances consumed by the user. In addition, the method can include determining, in real-time, whether the user is driving under an influence of the one or more consumed substances. In a number of embodiments, the method also can include updating a user profile of the user to include user driving behavior data comprising one or more of: (a) the sensor data, (b) the one or more consumed substances, or (c) a determination that the user is driving under influence of the one or more consumed substances.

In a number of embodiments, the method further can include determining, in real-time, a user location of the user based at least in part on a mobile device of the user. The method also can include, before receiving the sensor data, determining the one or more substance-detecting sensors from one or more airborne-particle sensors based on: (a) the user location, and (b) a respective sensor location of each of the one or more airborne-particle sensors.

In some embodiments, determining whether the user is driving under the influence of the one or more consumed substances can include: (a) determining a user motion of the user based on one or more telematics sensors on a mobile device of the user or a user vehicle of the user; (b) determining whether the user is driving based at least in part on the user motion; and/or (c) upon determining that the user is driving, determining whether the user is driving within a respective reaction time of at least one of the one or more consumed substances. In several embodiments, the method further can include determining a discount for an insurance premium for the user based at least in part on the user driving behavior data.

Various embodiments further can include a system comprising one or more processors; and one or more non-transitory computer-readable media storing computing instructions. In many embodiments, the computing instructions, when run on the one or more processors, can cause the one or more processors to perform one or more acts. The one or more acts can include receiving sensor data from one or more substance-detecting sensors located near or on a user. The one or more substance-detecting sensors can be configured to detect one or more chemical substances. The one or more chemical substances can include one or more inhalable cannabis-derived substances. In a number of embodiments, the one or more acts further can include determining, in real-time, a user location of the user based at least in part on a mobile device of the user. The one or more acts also can include, before receiving the sensor data, determining the one or more substance-detecting sensors from one or more airborne-particle sensors based on: (a) the user location, and (b) a respective sensor location of each of the one or more airborne-particle sensors.

In several embodiments, the one or more acts additionally can include determining, based on the sensor data, one or more consumed substances of the one or more chemical substances consumed by the user. In many embodiments, the one or more acts further can include determining, in real-time, whether the user is driving under an influence of the one or more consumed substances. In some embodiments, determining whether the user is driving under the influence of the one or more consumed substances can include: (a) determining a user motion of the user based on one or more telematics sensors on a mobile device of the user or a user vehicle of the user; (b) determining whether the user is driving based at least in part on the user motion; and/or (c) upon determining that the user is driving, determining whether the user is driving within a respective reaction time of at least one of the one or more consumed substances.

In a number of embodiments, the one or more acts also can include updating a user profile of the user to include user driving behavior data comprising one or more of: (a) the sensor data, (b) the one or more consumed substances, or (c) a determination that the user is driving under influence of the one or more consumed substances. The one or more acts further can include determining a discount for an insurance premium for the user based at least in part on the user driving behavior data.

Various embodiments further can include a non-transitory computer readable storage medium storing computing instructions, the computing instructions, when run on one or more processors, causing the one or more processors to perform one or more acts. The one or more acts can include receiving sensor data from one or more substance-detecting sensors located near or on a user. The one or more substance-detecting sensors can be configured to detect one or more chemical substances. The one or more chemical substances can include one or more inhalable cannabis-derived substances. In a number of embodiments, the one or more acts further can include determining, in real-time, a user location of the user based at least in part on a mobile device of the user. The one or more acts also can include, before receiving the sensor data, determining the one or more substance-detecting sensors from one or more airborne-particle sensors based on: (a) the user location, and (b) a respective sensor location of each of the one or more airborne-particle sensors.

In several embodiments, the one or more acts additionally can include determining, based on the sensor data, one or more consumed substances of the one or more chemical substances consumed by the user. In many embodiments, the one or more acts further can include determining, in real-time, whether the user is driving under an influence of the one or more consumed substances. In some embodiments, determining whether the user is driving under the influence of the one or more consumed substances can include: (a) determining a user motion of the user based on one or more telematics sensors on a mobile device of the user or a user vehicle of the user; (b) determining whether the user is driving based at least in part on the user motion; and/or (c) upon determining that the user is driving, determining whether the user is driving within a respective reaction time of at least one of the one or more consumed substances.

In a number of embodiments, the one or more acts also can include updating a user profile of the user to include user driving behavior data comprising one or more of: (a) the sensor data, (b) the one or more consumed substances, or (c) a determination that the user is driving under influence of the one or more consumed substances. The one or more acts further can include determining a discount for an insurance premium for the user based at least in part on the user driving behavior data.

### ADDITIONAL CONSIDERATIONS

Although remotely determining impaired driving caused by substances detectable by airborne-particle sensors has been described with reference to specific embodiments, it will be understood by those skilled in the art that various changes can be made without departing from the spirit or scope of the disclosure. Accordingly, the disclosure of embodiments is intended to be illustrative of the scope of the disclosure and is not intended to be limiting.

It is intended that the scope of the disclosure shall be limited only to the extent required by the appended claims. For example, to one of ordinary skill in the art, it will be readily apparent that any element of FIG. 1-4 can be modified, and that the foregoing discussion of certain of these embodiments does not necessarily represent a complete description of all possible embodiments. Additionally, one or more of the procedures, processes, operations, actions, and/or activities of the method in FIG. 4 can include different procedures, processes, actions, and/or activities and be performed by many different modules, in many different orders. As another example, the modules, models, elements, and/or systems within system 300 or system 310 in FIG. 3 can be interchanged or otherwise modified.

Replacement of one or more claimed elements constitutes reconstruction and not repair. Additionally, benefits, other advantages, and solutions to problems have been described with regard to specific embodiments. The benefits, advantages, solutions to problems, and any element or elements that can cause any benefit, advantage, or solution to occur or become more pronounced, however, are not to be construed as critical, required, or essential features or elements of any or all of the claims, unless such benefits, advantages, solutions, or elements are stated in such claim.

Moreover, embodiments and limitations disclosed herein are not dedicated to the public under the doctrine of dedication if the embodiments and/or limitations: (1) are not expressly claimed in the claims; and (2) are or are potentially equivalents of express elements and/or limitations in the claims under the doctrine of equivalents.

As will be appreciated based upon the foregoing specification, the above-described embodiments of the disclosure can be implemented using computer programming or engineering techniques including computer software, firmware, hardware or any combination or subset thereof. Any such resulting program, having computer-readable code means, can be embodied, or provided within one or more computer-readable media, thereby making a computer program product, e.g., an article of manufacture, according to the discussed embodiments of the disclosure. The computer-readable media can be, for example, but is not limited to, a fixed (hard) drive, diskette, optical disk, magnetic tape, semiconductor memory such as read-only memory (ROM), and/or any transmitting/receiving medium such as the Internet or other communication network or link. The article of manufacture containing the computer code can be made and/or used by executing the code directly from one medium, by copying the code from one medium to another medium, or by transmitting the code over a network.

These computer programs (also known as programs, software, software applications, “apps,” or code) include machine instructions for a programmable processor and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The “machine-readable medium” and “computer-readable medium,” however, do not include transitory signals. The term “machine-readable signal” refers to any signal used to provide machine instructions and/or data to a programmable processor.

As used herein, a processor can include any programmable system including systems using micro-controllers, reduced instruction set circuits (RISC), application specific integrated circuits (ASICs), logic circuits, and any other circuit or processor capable of executing the functions described herein. The above examples are example only and are thus not intended to limit in any way the definition and/or meaning of the term “processor.”

As used herein, the terms “software” and “firmware” are interchangeable and include any computer program stored in memory for execution by a processor, including RAM memory, ROM memory, EPROM memory, EEPROM memory, and non-volatile RAM (NVRAM) memory. The above memory types are example only and are thus not limiting as to the types of memory usable for storage of a computer program.

In one embodiment, a computer program is provided, and the program is embodied on a computer readable medium. In an exemplary embodiment, the system can be executed on a single computer system, without requiring a connection to a sever computer. In a further embodiment, the system is being run in a Windows® environment (Windows is a registered trademark of Microsoft Corporation, Redmond, Washington). In yet another embodiment, the system is run on a mainframe environment and a UNIX® server environment (UNIX is a registered trademark of X/Open Company Limited located in Reading, Berkshire, United Kingdom). The application is flexible and designed to run in various environments without compromising any major functionality. In some embodiments, the system includes multiple components distributed among a plurality of computing devices. One or more components can be in the form of computer-executable instructions embodied in a computer-readable medium. The systems and processes are not limited to the specific embodiments described herein. In addition, components of each system and each process can be practiced independent and separate from other components and processes described herein. Each component and process can also be used in combination with other assembly packages and processes.

As used herein, an element or step recited in the singular and preceded by the word “a” or “an” should be understood as not excluding plural elements, actions, operations, or steps, unless such exclusion is explicitly recited. Furthermore, references to “example embodiment” or “one embodiment” of the present disclosure are not intended to be interpreted as excluding the existence of additional embodiments that also incorporate the recited features.

The patent claims at the end of this document are not intended to be construed under 35 U.S.C. § 112 (f) unless traditional means-plus-function language is expressly recited, such as “means for” or “step for” language being expressly recited in the claim(s).

For simplicity and clarity of illustration, the drawing figures illustrate the general manner of construction, and descriptions and details of well-known features and techniques can be omitted to avoid unnecessarily obscuring the present disclosure. Additionally, elements in the drawing figures are not necessarily drawn to scale. For example, the dimensions of some of the elements in the figures can be exaggerated relative to other elements to help improve understanding of embodiments of the present disclosure. The same reference numerals in different figures denote the same elements.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements, but can include other elements not expressly listed or inherent to such process, method, system, article, device, or apparatus.

The terms “couple,” “coupled,” “couples,” “coupling,” and the like should be broadly understood and refer to connecting two or more elements mechanically and/or otherwise. Two or more electrical elements can be electrically coupled together, but not be mechanically or otherwise coupled together. Coupling can be for any length of time, e.g., permanent or semi-permanent or only for an instant. “Electrical coupling” and the like should be broadly understood and include electrical coupling of all types. The absence of the word “removably,” “removable,” and the like near the word “coupled,” and the like does not mean that the coupling, etc. in question is or is not removable.

As defined herein, “approximately” may, in some embodiments, mean within plus or minus ten percent of the stated value. In other embodiments, “approximately” can mean within plus or minus five percent of the stated value. In further embodiments, “approximately” can mean within plus or minus three percent of the stated value. In yet other embodiments, “approximately” can mean within plus or minus one percent of the stated value.

This written description uses examples to disclose the disclosure, including the best mode, and to enable any person skilled in the art to practice the disclosure, including making and using any devices or computer systems and performing any incorporated computer-based or computer-implemented methods. The patentable scope of the disclosure is defined by the claims, and can include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method being implemented via execution of computing instructions configured to run on one or more processors and stored on one or more non-transitory computer-readable media, the method comprising:
receiving sensor data from one or more substance-detecting sensors located near or on a user, wherein the one or more substance-detecting sensors are configured to detect one or more chemical substances;
determining, based on the sensor data, one or more consumed substances of the one or more chemical substances consumed by the user;
determining, in real-time, whether the user is driving under an influence of the one or more consumed substances; and
updating a user profile of the user to include user driving behavior data comprising one or more of: (a) the sensor data, (b) the one or more consumed substances, or (c) a determination that the user is driving under influence of the one or more consumed substances.

2. The method in claim 1, wherein:
the one or more chemical substances comprise one or more inhalable cannabis-derived substances.

3. The method in claim 1, further comprising:
determining, in real-time, a user location of the user based at least in part on a mobile device of the user.

4. The method in claim 1, further comprising:
before receiving the sensor data, determining the one or more substance-detecting sensors from one or more airborne-particle sensors based on: (a) a user location, and (b) a respective sensor location of each of the one or more airborne-particle sensors.

5. The method in claim 1, wherein determining whether the user is driving under the influence of the one or more consumed substances comprises:
upon determining that the user is driving, determining whether the user is driving within a respective reaction time of at least one of the one or more consumed substances.

6. The method in claim 5, wherein determining whether the user is driving under the influence of the one or more consumed substances further comprises:
determining a user motion of the user based on one or more telematics sensors on a mobile device of the user or a user vehicle of the user; and
determining whether the user is driving based at least in part on the user motion.

7. The method in claim 1, further comprising:
determining a discount for an insurance premium for the user based at least in part on the user driving behavior data.

8. A system comprising:
one or more processors; and
one or more non-transitory computer-readable media storing computing instructions that, when run on the one or more processors, cause the one or more processors to perform:
receiving sensor data from one or more substance-detecting sensors located near or on a user, wherein the one or more substance-detecting sensors are configured to detect one or more chemical substances;
determining, based on the sensor data, one or more consumed substances of the one or more chemical substances consumed by the user;
determining, in real-time, whether the user is driving under an influence of the one or more consumed substances; and
updating a user profile of the user to include user driving behavior data comprising one or more of: (a) the sensor data, (b) the one or more consumed substances, or (c) a determination that the user is driving under influence of the one or more consumed substances.

9. The system in claim 8, wherein:
the one or more chemical substances comprise one or more inhalable cannabis-derived substances.

10. The system in claim 8, wherein the computing instructions, when run on the one or more processors, further cause the one or more processors to perform:
determining, in real-time, a user location of the user based at least in part on a mobile device of the user.

11. The system in claim 8, wherein the computing instructions, when run on the one or more processors, further cause the one or more processors to perform:
before receiving the sensor data, determining the one or more substance-detecting sensors from one or more airborne-particle sensors based on: (a) a user location, and (b) a respective sensor location of each of the one or more airborne-particle sensors.

12. The system in claim 8, wherein determining whether the user is driving under the influence of the one or more consumed substances comprises:
upon determining that the user is driving, determining whether the user is driving within a respective reaction time of at least one of the one or more consumed substances.

13. The system in claim 12, wherein determining whether the user is driving under the influence of the one or more consumed substances further comprises:
determining a user motion of the user based on one or more telematics sensors on a mobile device of the user or a user vehicle of the user; and
determining whether the user is driving based at least in part on the user motion.

14. The system in claim 8, wherein the computing instructions, when run on the one or more processors, further cause the one or more processors to perform:
determining a discount for an insurance premium for the user based at least in part on the user driving behavior data.

15. One or more non-transitory computer readable storage media storing computing instructions, the computing instructions, when run on one or more processors, causing the one or more processors to perform operations comprising:
receiving sensor data from one or more substance-detecting sensors located near or on a user, wherein the one or more substance-detecting sensors are configured to detect one or more chemical substances;
determining, based on the sensor data, one or more consumed substances of the one or more chemical substances consumed by the user;
determining, in real-time, whether the user is driving under an influence of the one or more consumed substances; and
updating a user profile of the user to include user driving behavior data comprising one or more of: (a) the sensor data, (b) the one or more consumed substances, or (c) a determination that the user is driving under influence of the one or more consumed substances.

16. The one or more non-transitory computer readable storage media in claim 15, wherein:
the one or more chemical substances comprise one or more inhalable cannabis-derived substances.

17. The one or more non-transitory computer readable storage media in claim 15, wherein the computing instructions, when run on the one or more processors, further cause the one or more processors to perform:
determining, in real-time, a user location of the user based at least in part on a mobile device of the user; or
determining a discount for an insurance premium for the user based at least in part on the user driving behavior data.

18. The one or more non-transitory computer readable storage media in claim 15, wherein the computing instructions, when run on the one or more processors, further cause the one or more processors to perform:
before receiving the sensor data, determining the one or more substance-detecting sensors from one or more airborne-particle sensors based on: (a) a user location, and (b) a respective sensor location of each of the one or more airborne-particle sensors.

19. The one or more non-transitory computer readable storage media in claim 15, wherein determining whether the user is driving under the influence of the one or more consumed substances comprises:
upon determining that the user is driving, determining whether the user is driving within a respective reaction time of at least one of the one or more consumed substances.

20. The one or more non-transitory computer readable storage media in claim 19, wherein determining whether the user is driving under the influence of the one or more consumed substances further comprises:
determining a user motion of the user based on one or more telematics sensors on a mobile device of the user or a user vehicle of the user; and
determining whether the user is driving based at least in part on the user motion.
